(19) United States
(12) Patent Application Publication
(10) Pub. No.: US 2010/0092480 A1
(43) Pub. Date: Apr. 15, 2010

(54) MODULATORS OF PROTEIN PHOSPHATASE 2A

(75) Inventors: Yigong Shi, Plainsboro, NJ (US); Yongna Xing, Plainsboro, NJ (US)

Correspondence Address: PEPPER HAMILTON LLP, ONE MELLON CENTER, 50TH FLOOR, 500 GRANT STREET, PITTSBURGH, PA, 15219, US

(73) Assignee: THE TRUSTEES OF THE UNIVERSITY OF PRINCETON, Princeton, NJ (US)

(21) Appl. No.: 12/445,101

(22) PCT Filed: Oct. 12, 2007
(86) PCT No.: PCT/US07/81260

§ 371 Date: Dec. 15, 2009

(30) Foreign Application Priority Data
Oct. 13, 2006 (US) 60851554

(51) Int. Cl. A61K 39/395; C07K 16/40; A61P 35/04; G06F 19/00

(52) U.S. Cl. 424/146.1; 530/388.2; 702/27

(57) ABSTRACT

Atomic coordinates for human serine/threonine protein phosphotase 2A (PP2A) core, as well as methods for using these atomic coordinates to prepare inhibitors of PP2A and inhibitors prepared using such methods are provided herein. A biochemical analysis of the interactions of PP2A core is also provided. Compositions including mimetics and small molecules of the invention and, optionally, secondary agents may be used to treat disorders in which PP2A activity plays a contributing role.

### CROSS-REFERENCE TO RELATED APPLICATIONS

This application claims priority to and benefit of U.S. Provisional Application No. 60/851,554, entitled “Structure of Protein Phosphatase 2A Core Enzyme Bound to Tumor-Inducing Toxins”, filed on Oct. 13, 2006: the entire contents of which are hereby incorporated by reference in its entirety.

### GOVERNMENT INTERESTS

Not applicable

### PARTIES TO A JOINT RESEARCH AGREEMENT

Not applicable

### INCORPORATION BY REFERENCE OF MATERIAL SUBMITTED ON A COMPACT DISC

Not applicable

### BACKGROUND

1. Field of Invention

The invention presented herein provides compositions and methods for modulation of protein phosphatase 2A.

2. Description of Related Art

Reversible protein phosphorylation is a fundamental regulatory mechanism in all aspects of biology. Protein phosphatase 2A (PP2A) is a major serine/threonine phosphatase involved in many aspects of cellular function including, for example, cell cycle regulation, cell growth control, development, regulation of multiple signal transduction pathways, cytoskeleton dynamics, and cell mobility. Additionally, PP2A is also an important tumor suppressor protein.

PP2A is made up of at least three subunits (FIG. 1A). The PP2A core is made up of a catalytic (C) subunit and a scaffold (A) subunit. The C- and A-subunits each have two isoforms in mammalian cells, α and β, which share significant sequence similarity. Although in both cases, the α isoform is more abundant than the β isoform. PP2A core interacts with a third regulatory (B) subunit to form a hetero-trimeric holoenzyme. B-subunits have been separated into four subfamilies: B (or PR55), B′ (or B56 or PR61), B″ (or PR72), and B′″ (or PR93/PR110), with at least 16 members in each subfamily and appear to determine substrate specificity as well as the spatial and temporal functions of PP2A. The expression level of various types of B-subunits has been shown to be highly diverse depending upon cell types and tissues.

PP2A core is found in most cells at relatively abundant concentrations which may indicate a more significant role for PP2A core than merely being an intermediate for PP2A holoenzyme. Moreover, PP2A core is regulated by numerous cellular regulatory proteins. For example, the carboxy-terminal, Leu309, of PP2A C-subunit is methylated by a specific leucine carboxyl methyltransferase (LCMT), and this methylation allows PP2A core to interact with the regulatory B subunit to form the PP2A holoenzyme (FIG. 1A). Additionally, a fully methylated PP2A core is a substrate for a PP2A-specific methyl esterase (PME), which specifically removes the methyl group from Leu309 of the C-subunit (FIG. 1A). The phosphatase activity and specificity of PP2A core also appear to be regulated by phosphatase 2A phosphatase activator (PTPA).

Inactivation of both the α and β isoforms of the PP2A core A-subunit has been linked to several forms of cancer. For example, mutations in the A-subunit that result in compromised binding to the B-subunit or C-subunit or that substantially reduce binding between the B- and C-subunits and the A-subunit have been associated with a variety of primary human tumors types. In addition, an N-terminally truncated B subunit mutant (B′γ1) has been shown to be associated with an increased metastasis in melanoma cells. Additionally, PP2A core has been shown to be a target for several known carcinogens, such as, for example, okadaic acid (OA) and microcystin-LR (MCLR), and these carcinogens may act by specifically inactivating PP2A core. For example, OA has been shown to exhibit an inhibitory constant (IC₅₀) that is significantly greater for PP2A (approximately 0.1 nM) than other related phosphatases, such as PP1 (10 nM), and MCLR has also been shown to potently inhibit the activity of PP2A.

### BRIEF SUMMARY OF THE INVENTION

Embodiments of the invention described herein are generally directed to PP2A core binding compounds, methods for preparing PP2A core binding compounds, pharmaceutical compositions derived from such compounds, and methods for identifying carcinogens.

Various embodiments include a protein phosphatase 2A (PP2A) binding compound including a molecule having a three-dimensional structure corresponding to atomic coordinates derived from at least a portion of an atomic model of protein phosphatase 2A (PP2A) core having okadaic acid or microcystin-LR bound thereto wherein the compound is not okadaic acid or microcystin.

In some embodiments, the molecule may be an inhibitor of protein phosphatase 2A (PP2A). In other embodiments, the molecule may have a three-dimensional structure corresponding to atomic coordinates of at least a portion of okadaic acid, microcystin-LR or a combination thereof bound to protein phosphatase 2A (PP2A) core, and the compound may interact with the catalytic (C) subunit of protein phosphatase 2A (PP2A) core that correspond to at least a portion of the interactions observed between the catalytic (C) subunit of protein phosphatase 2A (PP2A) core and okadaic acid or microcystin-LR. In certain embodiments, the molecule may bind protein phosphatase 2A (PP2A) at a binding site for okadaic acid and microcystin-LR on the catalytic (C) subunit of PP2A core. In certain other embodiments, the molecule may bind to a portion of the catalytic (C) subunit of protein phosphatase 2A (PP2A) core comprising at least a portion of amino acids 25-288 of the catalytic (C) subunit.

In various other embodiments, the molecule may have=a shape, a charge, a size or combinations thereof substantially corresponding to a portion of protein phosphatase 2A (PP2A) core. In some embodiments, the molecule may bind to a catalytic (C) subunit of protein phosphatase 2A (PP2A) core or a scaffolding (A) subunit of protein phosphatase 2A (PP2A) core at an interface between the catalytic (C) subunit and the scaffolding (A) subunit. In other embodiments, the molecule may correspond to a portion of the catalytic (C) subunit of protein phosphatase 2A (PP2A) core comprising at least a portion of amino acids 24-115, 258-294 or a combination thereof of the catalytic (C) subunit, and in still other embodiments, the molecule may correspond to a portion of a the scaffolding (A) subunit of protein phosphatase 2A (PP2A) core comprising at least a portion of HEAT repeats 11-15.

In certain embodiments, the molecule may have a shape, a charge, a size or combinations thereof substantially complementary to a portion of protein phosphatase 2A (PP2A) core. In some embodiments, the molecule may be substantially complementary to a portion of a scaffolding (A) subunit of protein phosphatase 2A (PP2A) core, and in others, the molecule may bind to a scaffolding (A) subunit of PP2A core and inhibits flexibility of the scaffolding (A) subunit. In certain other embodiments, the molecule may be substantially complementary to a portion of a catalytic (C) subunit of protein phosphatase 2A (PP2A) core corresponding to a region of the catalytic (C) subunit where phosphatase 2A phosphatase activator (PTPA) binds, and in others, the molecule may inhibit modulation of PP2A by phosphatase 2A phosphatase activator (PTPA).

The molecule of some embodiments may bind to protein phosphatase 2A (PP2A) core with a greater affinity than a naturally occurring substrate, and in others, the molecule may inhibit protein phosphatase 2A (PP2A) catalyzed tyrosine phosphorylation, serine phosphorylation or a combination thereof.

In various embodiments, the composition may include a pharmaceutically acceptable excipient or carrier.

Various other embodiments include a method for preparing a protein phosphatase 2A (PP2A) core binding compound including the steps of applying a three-dimensional molecular modeling algorithm to the atomic coordinates of at least a portion of protein phosphatase 2A (PP2A) core, a catalytic (C) subunit of protein phosphatase 2A (PP2A) core, or a scaffolding (A) subunit of protein phosphatase 2A (PP2A) core; determining spatial coordinates of the at least a portion of protein phosphatase 2A (PP2A) core; electronically screening stored spatial coordinates of candidate compounds against the spatial coordinates of the at least a portion of protein phosphatase 2A (PP2A) core; and identifying candidate compounds that bind to protein phosphatase 2A (PP2A) core.

The method of some embodiments may also include the step of identifying a molecule has a shape, a charge, a size or combinations thereof substantially complementary to a portion of protein phosphatase 2A (PP2A) core, the catalytic (C) subunit of protein phosphatase 2A (PP2A) core, or the scaffolding (A) subunit of protein phosphatase 2A (PP2A) core. Particular embodiments may include the step of identifying candidate compounds that deviate from the atomic coordinates of the at least a portion of protein phosphatase 2A (PP2A) core, the catalytic (C) subunit of protein phosphatase 2A (PP2A) core, or the scaffolding (A) subunit of protein phosphatase 2A (PP2A) core by a root mean square deviation of less than about 10 angstroms. Methods of some embodiments may also include the step of testing identified candidate compounds for binding protein phosphatase 2A (PP2A) core, and other embodiments, may include testing identified candidate compounds for inhibiting protein phosphatase 2A (PP2A) core activity. In still other embodiments, the method may include the step of identifying candidate compounds having a binding affinity for protein phosphatase 2A (PP2A) core, the catalytic (C) subunit of protein phosphatase 2A (PP2A) core, or the scaffolding (A) subunit of protein phosphatase 2A (PP2A) core greater than a naturally occurring substrate. Certain embodiments of the method may include the step of identifying candidate compounds that inhibit tyrosine phosphorylation, serine phosphorylation or a combination thereof catalyzed by protein phosphatase 2A (PP2A) core.

In particular embodiments, the atomic coordinates of at least a portion of the protein phosphatase 2A (PP2A) core or the catalytic (C) subunit of protein phosphatase 2A (PP2A) core may include okadaic acid or microcystin-LR bound to the protein phosphatase 2A (PP2A) core or the catalytic (C) subunit, and electronically screening may include electronically screening stored spatial coordinates of candidate compounds against atomic coordinates of okadaic acid or microcystin-LR bound to the protein phosphatase 2A (PP2A) core or the catalytic (C) subunit.

Other embodiments of the invention are directed to a pharmaceutical composition comprising an effective amount of a compound prepared by the method including the steps of: applying a three-dimensional molecular modeling algorithm to the atomic coordinates of at least a portion of protein phosphatase 2A (PP2A) core, a catalytic (C) subunit of protein phosphatase 2A (PP2A) core, or a scaffolding (A) subunit of protein phosphatase 2A (PP2A) core, determining spatial coordinates of at least a portion of the protein phosphatase 2A (PP2A) core, the catalytic (C) subunit of protein phosphatase 2A (PP2A) core, or the scaffolding (A) subunit of protein phosphatase 2A (PP2A) core, electronically screening stored spatial coordinates of candidate compounds against the spatial coordinates of at the least a portion of the protein phosphatase 2A (PP2A) core, the catalytic (C) subunit of protein phosphatase 2A (PP2A) core, or the scaffolding (A) subunit of protein phosphatase 2A (PP2A) core, and identifying compounds that mimic the structure of the at least a portion of the protein phosphatase 2A (PP2A) core, the catalytic (C) subunit of protein phosphatase 2A (PP2A) core, or the scaffolding (A) subunit of protein phosphatase 2A (PP2A) core; and a pharmaceutically effective excipient or carrier.

In some embodiments the pharmaceutical composition may include a molecule that bind to protein phosphatase 2A (PP2A) core, the catalytic (C) subunit of protein phosphatase 2A (PP2A) core, or the scaffolding (A) subunit of protein phosphatase 2A (PP2A) core. In others, the atomic coordinates of at least a portion of the protein phosphatase 2A (PP2A) core or the catalytic (C) subunit of protein phosphatase 2A (PP2A) core may include okadaic acid or microcystin-LR bound to the protein phosphatase 2A (PP2A) core or the catalytic (C) subunit and electronically screening may include electronically screening stored spatial coordinates of candidate compounds against atomic coordinates of okadaic acid or microcystin-LR bound to the protein phosphatase 2A (PP2A) core or the catalytic (C) subunit.

Still other embodiments described herein include a method for identifying a carcinogen comprising determining the atomic coordinates of a compound, applying a three-dimensional molecular modeling algorithm to the atomic coordinates of the compound, applying a three-dimensional molecular modeling algorithm to atomic coordinates of at least a portion of protein phosphatase 2A (PP2A) core, a catalytic (C) subunit of protein phosphatase 2A (PP2A) core, a scaffolding (A) subunit of protein phosphatase 2A (PP2A) core, okadaic acid bound to PP2A core or microcystin-LR bound to PP2A core, electronically screening atomic coordinates of the compound against the atomic coordinates of at the least a portion of the protein phosphatase 2A (PP2A) core, the catalytic (C) subunit of protein phosphatase 2A (PP2A) core, the scaffolding (A) subunit of protein phosphatase 2A (PP2A) core, okadaic acid bound to PP2A core or microcystin-LR bound to PP2A core, and identifying the compound as a carcinogen if the compound is substantially similar to the structure of the at least a portion of the protein phosphatase 2A (PP2A) core, the catalytic (C) subunit of protein phosphatase 2A (PP2A) core, the scaffolding (A) subunit of protein phosphatase 2A (PP2A) core, okadaic acid bound to PP2A core or microcystin-LR bound to PP2A core.

In some embodiments, the identified compound may deviate from the atomic coordinates of the at least a portion of protein phosphatase 2A (PP2A) core, the catalytic (C) subunit of protein phosphatase 2A (PP2A) core, the scaffolding (A) subunit of protein phosphatase 2A (PP2A) core, okadaic acid bound to PP2A core or microcystin-LR bound to PP2A core by a root mean square deviation of less than about 10 angstroms.

In other embodiments, the method may further include testing identified compounds for binding protein phosphatase 2A (PP2A) core, and in still other embodiments, the method may include the step of testing identified compounds for inhibiting protein phosphatase 2A (PP2A) core activity.

In various embodiments, the method may include the step of identifying compounds that inhibit tyrosine phosphorylation, serine phosphorylation or a combination thereof catalyzed by protein phosphatase 2A (PP2A) core, and in certain embodiments, the step of electronically screening may include electronically screening stored spatial coordinates of an identified compound against atomic coordinates of unbound okadaic acid or microcystin-LR.

### DESCRIPTION OF DRAWINGS

For a fuller understanding of the nature and advantages of the present invention, reference should be made to the following detailed description taken in connection with the accompanying drawings. All figures where structural representations are shown were prepared using MOLSCRIPT (Kraulis (1991) J Appl Crystallogr 24:946-950) and GRASP (Nicholls et al. (1991) Proteins: Struct Fund Genet 11:281-296).

FIG. 1A is a schematic diagram describing the PP2A system.

FIG. 1B shows an overall structure of okadaic acid (OA) bound PP2A core. The catalytic and scaffolding subunits are labeled and the lower panel shows a perspective that is rotated 90° from the upper panel.

FIG. 1C shows a stereoscopic view of OA bound to the catalytic (C) subunit of PP2A.

FIG. 1D shows an overall structure of microcystin-LR (NICER) bound PP2A core (top panel), and a stereoscopic view of MCLR bound to the C-subunit of PP2A (lower panel).

FIG. 2A shows an alignment of PP2A C-subunit α isoform and PP2A C-subunit β isoform compared to other serine/threonine phosphotase proteins: PP1, PP2B, PP4, PP5, PP6, and PP7. Secondary structural elements are provided above the primary sequence alignment. Residues that form hydrogen bonds with the A-subunit are identified by light gray and dark gray circles, and residues that contribute to van der Waals contacts with the A-subunit are identified with dark gray squares. Residues that form hydrogen bonds with OA and NICER are identified by dark gray triangles, and residues that contribute to van der Waals contacts are identified by light gray triangles.

FIG. 2B shows an overlay of the PP2A C-subunit structure (gray) with the structures of PP1 (PDB code 1FJM, light gray) and PP5 (PDB code 1S95, dark gray). OA is shown in black.

FIG. 3A shows the PP2A C-subunit interacting with HEAT repeats 11-15 of the A-subunit (left panel) and the same rotated 90° (right panel).

FIG. 3B shows a stereoscopic representation of PP2A C-subunit (light side chains) and HEAT repeats 11 and 12 of PP2A A-subunit (dark side chains) with hydrogen bonds represented by red dotted lines.

FIG. 3C shows a stereoscopic representation of PP2A C-subunit (light side chains) and HEAT repeats 13-15 of PP2A A-subunit (medium side chains) with hydrogen bonds represented by dotted lines.

FIG. 4A shows a OA (light gray) bound to PP2A C-subunit (gray) in the left panel, and a detailed view of the interface between OA and PP2A C-subunit (right panel). Mn atoms are shown as spheres and hydrogen bonds are represented by dotted lines.

FIG. 4B shows a NICER (light gray) bound to PP2A C-subunit (gray) in the left panel, and a detailed view of the interface between OA and PP2A C-subunit (right panel). Mn atoms are shown as spheres and hydrogen bonds are represented by dotted lines.

FIG. 4C shows an overlay of OA bound to PP2A C-subunit (dark gray) and PP1 (light gray).

FIG. 4D shows a transparent mesh representation of OA bound PP2A (upper panel) and a transparent mesh representation of OA bound PP1 (lower panel). Mn atoms are shown as spheres.

FIG. 5A shows an overlay of free A-subunit (light gray) and A-subunit from the PP2A core structure (dark gray).

FIG. 5B shows an overlay of HEAT repeats 13-15 of free A-subunit (light gray) and HEAT repeats 13-15 of A-subunit from the PP2A core (dark gray).

FIG. 5C shows an alignment of one HEAT repeat (wire) with the HEAT next repeat (cylinder).

FIG. 5D shows a stereoscopic view of HEAT repeats 12 and 13 of free A-subunit (left panel) and PP2A core (right panel).

FIG. 6A shows a western blot of PP2A core using an antibody that recognizes unmethylated C-subunit (NaOH removes the methyl group, left panel), the results of gel filtration chromatography (middle panel), and SDS-PAGE of fractions containing A-, B″- and C-subunits (right panel).

FIG. 6B shows a model of PP2A core with a B-subunit bound to the N-terminus of the A-subunit where flexibility within the A-subunit (indicated by arrows) allows the C- and B-subunits to contact one another.

### DETAILED DESCRIPTION

It must be noted that, as used herein and in the appended claims, the singular forms “a,” “an,” and “the” include plural reference unless the context clearly dictates otherwise. Unless defined otherwise, all technical and scientific terms used herein, have the same meanings as commonly understood by one of ordinary skill in the art. Although any methods similar or equivalent to those described herein can be used in the practice or testing of embodiments of the present invention, the preferred methods are now described. All publications and references mentioned herein are incorporated by reference. Nothing herein is to be construed as an admission that the invention is not entitled to antedate such disclosure by virtue of prior invention.

As used herein, the term “about” means plus or minus 10% of the numerical value of the number with which it is being used. Therefore, about 50% means in the range of 45%-55%.

The terms “mimetic,” “peptide mimetic,” and “peptidomimetic” are used interchangeably herein, and generally refer to a peptide, partial peptide or non-peptide molecule that mimics the tertiary binding structure or activity of a selected native peptide or protein functional domain (e.g., binding motif or active site). These peptide mimetics include recombinantly or chemically produced peptides, recombinantly or chemically modified peptides, as well as non-peptide agents, such as small molecule drug mimetics as further described below. Mimetic compounds can have additional characteristics that enhance their therapeutic application, such as increased cell permeability, greater affinity and/or avidity, and prolonged biological half-life.

As used herein, the terms “pharmaceutically acceptable,” “physiologically tolerable,” and grammatical variations thereof, as they refer to compositions, carriers, diluents, and reagents, are used interchangeably and represent that the materials are capable of administration upon a mammal without the production of undesirable physiological effects such as nausea, dizziness, rash, or gastric upset.

“Providing,” when used in conjunction with a therapeutic, means to administer a therapeutic directly into or onto a target tissue, or to administer a therapeutic to a patient whereby the therapeutic positively impacts the tissue to which it is targeted.

As used herein, “subject,” “patient” or “individual” refers to an animal or mammal including, but not limited to, a human, dog, cat, horse, cow, pig, sheep, goat, chicken, monkey, rabbit, rat, or mouse, etc.

As used herein, the term “therapeutic” means an agent utilized to treat, combat, ameliorate, prevent or improve an unwanted condition or disease of a patient. Embodiments of the present invention are directed to promote apoptosis and thus, cell death.

The terms “therapeutically effective amount” or “effective amount,” as used herein, may be used interchangeably and refer to an amount of a therapeutic compound component of the present invention. For example, a therapeutically effective amount of a therapeutic compound is a predetermined amount calculated to achieve the desired effect, i.e., to effectively modulate the activity of protein phosphatase 2A (PP2A).

“Inhibitor” means a compound which reduces or prevents a particular interaction or reaction. For example, an inhibitor may bind to PP2A C-subunit inactivating the C-subunit and inhibiting the phosphotyrosyl activity of PP2A.

“Pharmaceutically acceptable salts” include both acid and base addition salts. “Pharmaceutically acceptable acid addition salt” refers to those salts which retain the biological effectiveness and properties of the free bases and which are not biologically or otherwise undesirable and formed with inorganic acids, such as hydrochloric acid, hydrobromic acid, sulfuric acid, nitric acid, carbonic acid, phosphoric acid, and the like. Organic acids may be selected from aliphatic, cycloaliphatic, aromatic, araliphatic, heterocyclic, carboxylic, and sulfonic classes of organic acids, such as formic acid, acetic acid, propionic acid, glycolic acid, gluconic acid, lactic acid, pyruvic acid, oxalic acid, malic acid, maleic acid, maloneic acid, succinic acid, fumaric acid, tartaric acid, citric acid, aspartic acid, ascorbic acid, glutamic acid, anthranilic acid, benzoic acid, cinnamic acid, mandelic acid, embonic acid, phenylacetic acid, methanesulfonic acid, ethanesulfonic acid, p-toluenesulfonic acid, salicyclic acid, and the like.

The invention described herein is generally directed to atomic coordinates defining PP2A core, methods for using the atomic coordinates of PP2A core, mimetics and small molecules prepared using such methods, and pharmaceutical compositions made from mimetics and small molecules so prepared.

The atomic coordinates of PP2A core were provided from a full-length α-isoform C-subunit and a full-length α-isoform A-subunit. The C-subunit was overexpressed in baculovirus-infected insect cells and purified to homogeneity by affinity chromatography. The B-subunit was expressed in bacteria as a glutathione S-transferase (GST) fusion protein and immobilized on glutathione resin. PP2A core was assembled by capturing purified C-subunit on the immobilized B-subunit. The PP2A core complex was then released from the glutathione resin by cleavage with thrombin. The assembled complex was further purified using ion exchange chromatography. Assembled, purified PP2A core was next shown to exhibit catalytic activity that is identical to the catalytic activity of PP2A core purified from bovine brain using phosphorylase A as the substrate. Additionally, the phosphatase activity of the assembled, purified PP2A core was efficiently inhibited by stoichiometric amounts of either okadaic acid (OA) or microcystin-LR (MCLR).

Before crystallization, assembled, purified PP2A core was incubated with either 1.2 molar equivalence of OA or MCLR, and crystals of PP2A core separately bound to either OA or MCLR were obtained. Crystallographic structures for these crystals were determined by molecular replacement and refined to 2.6 Å resolution for OA bound PP2A core and 2.8 Å resolution for MCLR bound PP2A core (Table 1).

TABLE 1
Crystallographic Data Collection Statistics

| | PP2A + OA | PP2A + MCLR |
|---|---|---|
| Beamline | NSLS-X25 | NSLS-X25 |
| Space group | 1222 | 1222 |
| Resolution (Å) | 50.0-2.5 Å | 50.0-2.8 Å |
| Total observations | 299,020 | 238,497 |
| Unique observations | 62,598 | 44,149 |
| Data coverage (outer shell) | 99.4% (100.0%) | 98.3% (97.0%) |
| R_{sym} (outer shell) | 0.070 (0.465) | 0.061 (0.400) |
| Refinement | | |
| Resolution range (Å) | 30.0-2.6 Å | 30.0-2.8 Å |
| Number of reflections (\|F\| > 0) | 53,380 | 42,363 |
| Data coverage | 94.9% | 92.9% |
| R_{work}/R_{free} | 0.226/0.270 | 0.222/0.264 |
| Total number of atoms | 6,988 | 6,956 |
| Number of waters | 64 | 22 |
| Rmsd bond length (Å) | 0.010 | 0.010 |
| Rmsd bond angles (°) | 1.60 | 1.53 |
| Ramachandran Plot | | |
| Most favored (%) | 87.5 | 86.3 |
| Additionally allowed (%) | 11.2 | 12.2 |
| Generously allowed (%) | 1.1 | 1.3 |
| Disallowed (%) | 0.2 | 0.2 |

R_{sym} = Σₕ Σᵢ/I_{h,i} − Iₕ/IΣₕΣᵢI_{h,i}, where Iₕ is the mean intensity of the, observations of symmetry-related reflections of h.
R = Σ|F_{obs} − F_{calc}|/ΣF_{obs.} where F_{obs} = F_{P} and F_{calc} is the calculated protein structure factor from the atomic model.
R_{free} was calculated with 5% of the reflections.
Rmsd bond lengths and angles are the deviations from ideal values.

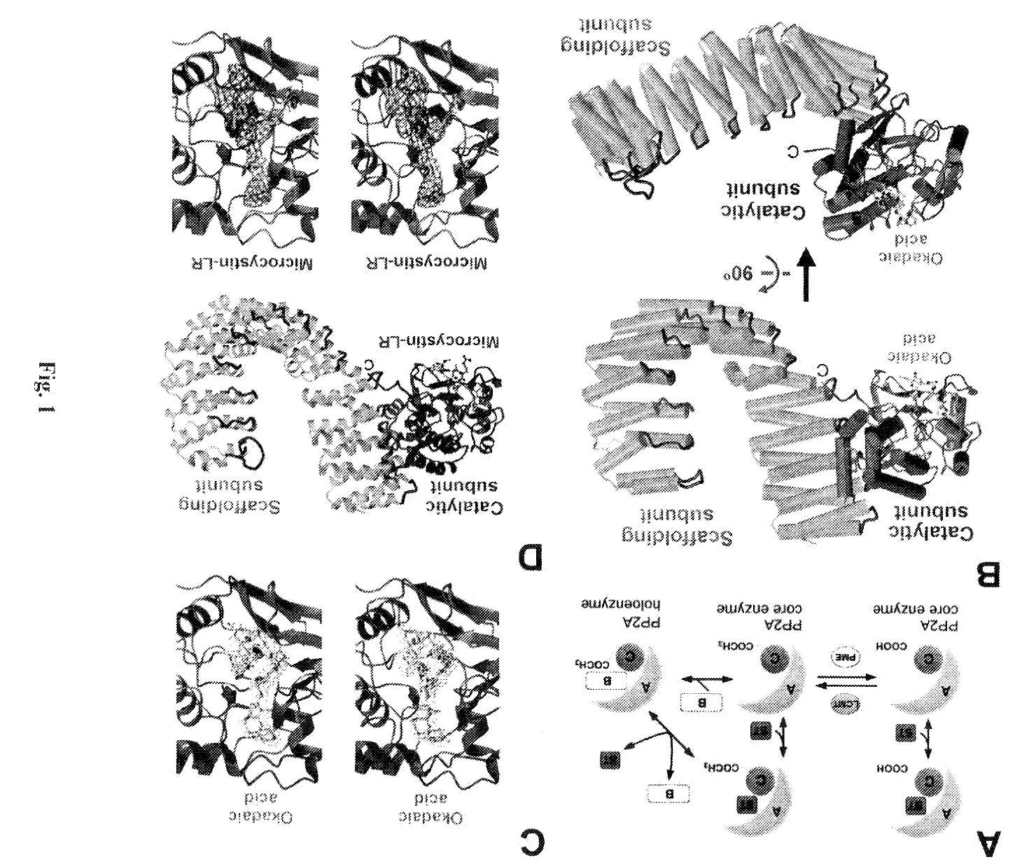

The structure of PP2A core OA and PP2A core MCLR co-crystals exhibit an extended architecture, measuring about 130 Å in length, about 80 Å in height, and about 60 Å in width (FIG. 1B). The A-subunit forms an elongated, horseshoe-shaped structure characterized by double-layered α helices made up of 15 HEAT repeats with each HEAT repeat including a pair of antiparallel α helices. The inter-helical region within each HEAT repeat forms a contiguous ridge (hereafter “the ridge”) that may bind substrate. The C-subunit appears to bind to one end of the scaffold through interactions with the ridge of HEAT repeats 11-15 (FIG. 1B), and interactions between the B- and C-subunits appear to result in burial of approximately 2,070 Å² of otherwise exposed surface area.

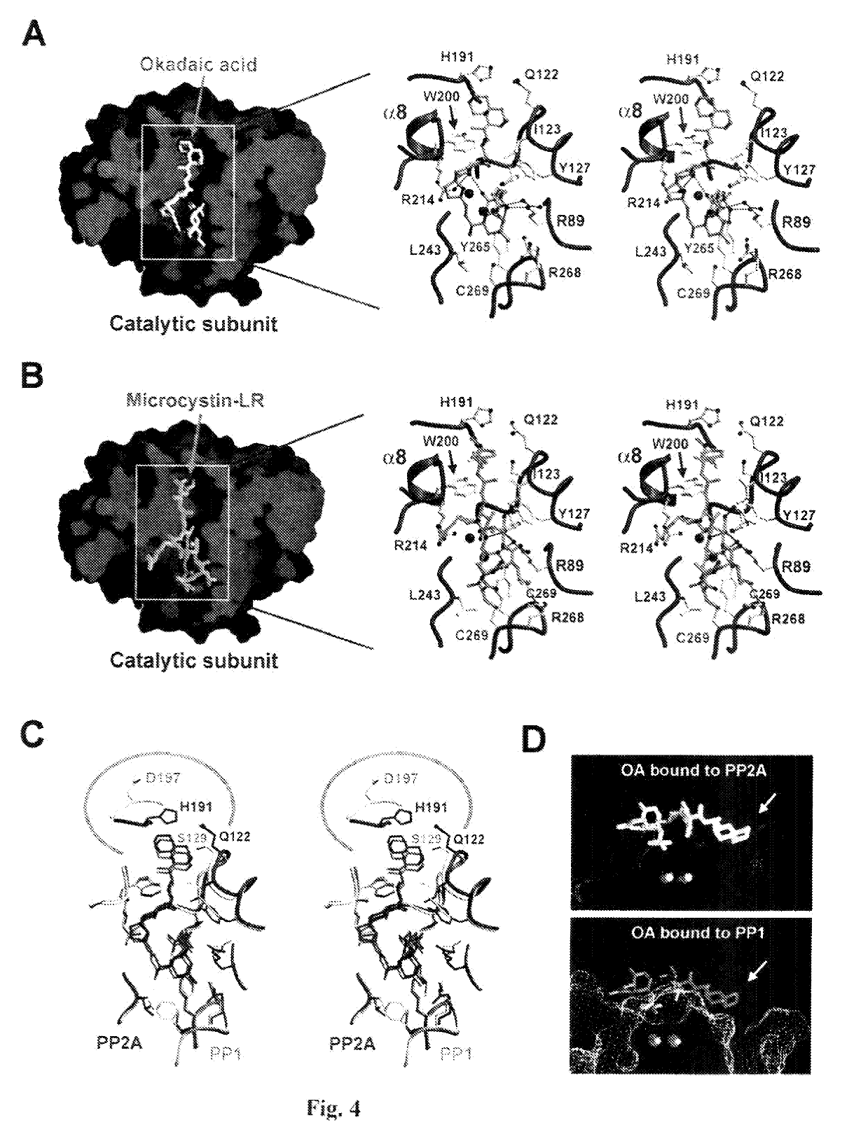

The C-subunit itself adopts an α/β fold typical of PPP family phosphatases and contains two active site metal atoms (FIG. 1B, FIG. 4A and FIG. 4B) which were determined to be manganese (Mn) using inductively coupled plasma-emission spectrometry element. The carboxy terminus of the C-subunit appear to extend towards the amino terminus of the A-subunit opposite the site of the C-subunit binding to the A-subunit. OA and MCLR appear to bind the C-subunit on the side of the C-subunit facing away from the C-subunit/B-subunit interface. The binding sites for OA (FIG. 1C) and MCLR (FIG. 1D) almost completely overlap, and a similar set of C-subunit amino acids may be involved in interactions with both toxins.

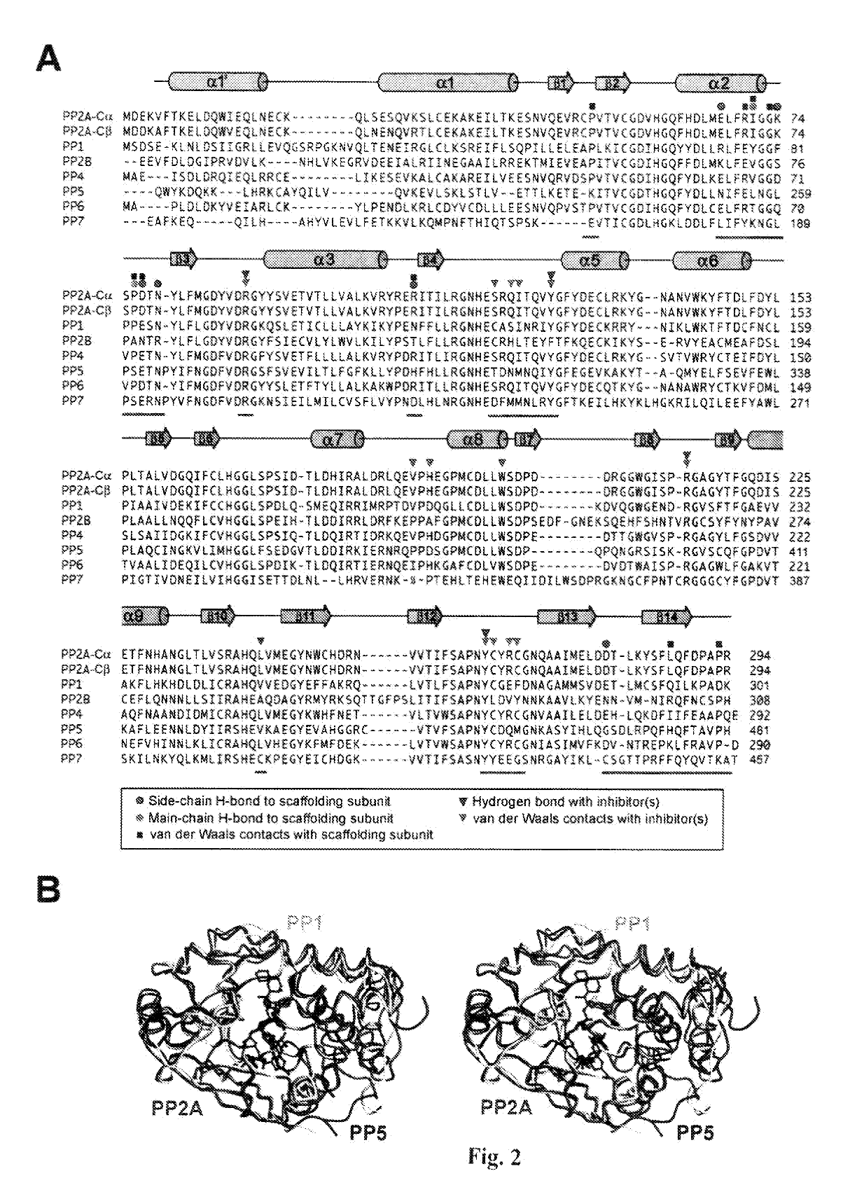

FIG. 2A is a sequence comparison of the α and β isoforms of PP2A C-subunits with a number of related protein serine/threonine phosphatases: PP1, PP2B, PP4, PP5, PP6, and PP7, and shows that PP2A core shares significant sequence similarity with each of these phosphatases. FIG. 2A also provides the secondary structural elements associated with PP2A C-subunit and amino acid residues involved in contacting the A-subunit of PP2A denoted by circles and squares above the primary sequence and amino acid residues involved in contacting OA and MCLR are denoted by triangles. It is of note that many of the residues involved in contacting both A-subunits and OA and MCLR are conserved among the aligned sequences. Moreover, the structures of several related protein serine/threonine phosphatases including, for example, PP1 and PP5, have been determined. Although the C-subunit of PP2A core appears to structurally resemble these related phosphatases as shown in the overlay provided in FIG. 2B, there are significant structural differences primarily in solvent-exposed surface loops resulting in a relatively large root-mean-square-deviation (RMSD). For example, the RMSD for PP2A C-subunit when compared to PP1 C-subunit is about 3.40 Å over all 284 aligned Cα atoms. Without wishing to be bound by theory, these structural differences may result in functional differences between PP2A and other related phosphatases.

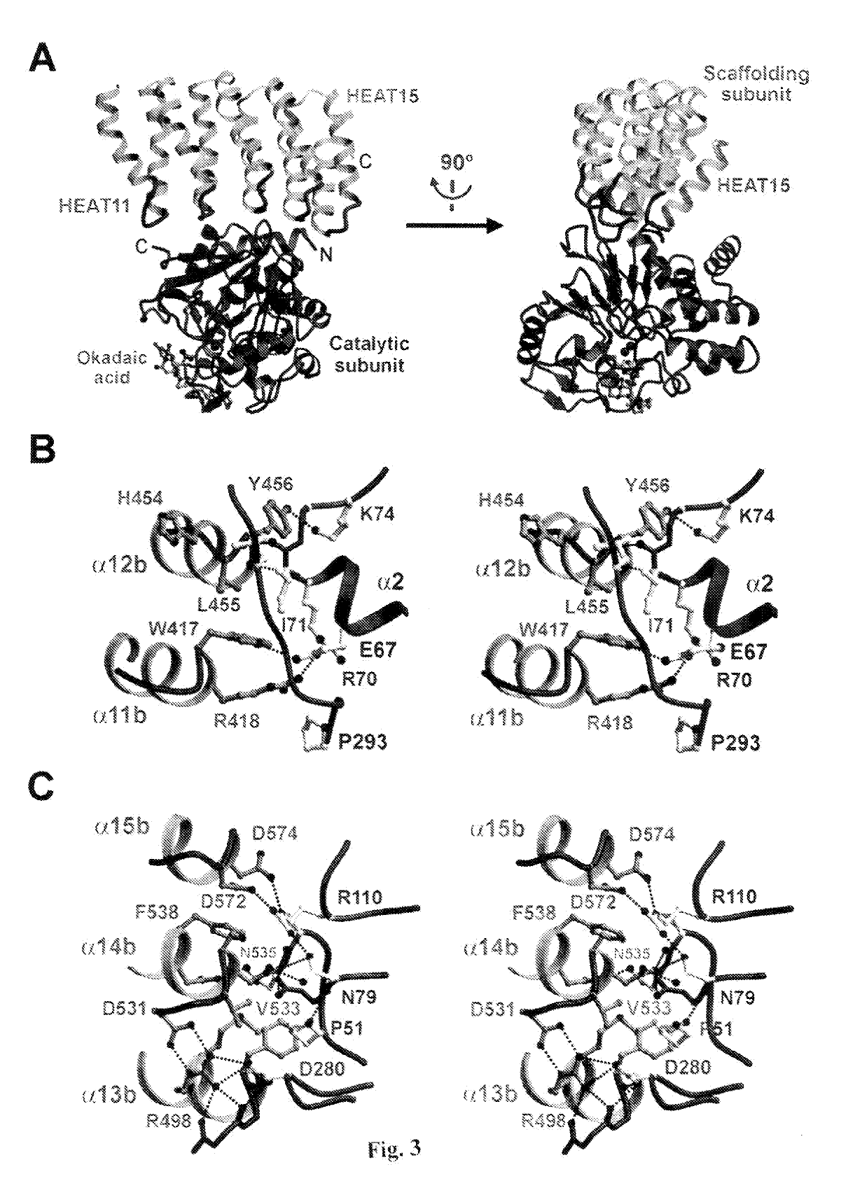

PP2A core maintains strong and specific interactions between the C- and A-subunits despite the significant sequence similarity between the C-subunit of PP2A and the catalytic subunits of other serine/threonine phosphatases as described above. As illustrated in FIG. 3A, the interface between the C-subunit and the A-subunit primarily involves HEAT repeats 11-15 in the A-subunit and the region surrounding helix α2 and the c-terminus of the C-subunit. Recognition specificity appears to be provided by about 15 inter-molecular hydrogen bonds and several van der Waals contacts in this region of the complex which are shown in FIG. 3B and FIG. 3C. The linear arrangement of HEAT repeats allows the entire interface to be divided into two segments. In one segment, Trp417 from HEAT repeat 11 and Leu455 from HEAT repeat 12 of the A-subunit make multiple van der Waals contacts to Arg70 and Ile71 of helix α2 of the C-subunit. At the periphery, four hydrogen bonds between the side chains of Arg418 of the HEAT repeat 11 of the A-subunit and Glu67 of the C-subunit further strengthen C-subunit/A-subunit interactions (FIG. 3B). Interestingly, a mutation to Arg418 to Trp in the A-subunit of melanoma-derived cDNA may negatively impact interactions with the C-subunit. In the other segment, HEAT repeats 13, 14, and 15 of the A-subunit for inter- and intra-molecular hydrogen bonds forming two extensive networks. For example, in one network, the carboxylate side chain of Asp280 of the C-subunit makes four inter-molecular hydrogen bonds: one to the carbonyl oxygen atom of Pro493, one to the backbone amide of Tyr495, and two to the side chain of Arg498 of the A-subunit. These interactions appear to be buttressed by four intra-molecular hydrogen bonds to the side chain of Arg498 of the A-subunit: a pair of charge-stabilized contacts from the carboxylate side chain of Asp531 and two additional contacts from the carbonyl groups of Asp492 and Pro493 (FIG. 3C). In another network, the side chain of Asn535 of the A-subunit makes four inter-molecular hydrogen bonds: two to the side chain of Asn79 and two to the carbonyl oxygen atoms of Phe76 and Thr77 of the C-subunit (FIG. 3C). Val533 of the A-subunit resides at the center of this portion of the interface and appears to interact with Pro51 in the C-subunit. It is of note that Val533 (Val454 in the β isoform) is mutated to Ala in some forms of colon cancer, which is consistent with the observation that a V545A mutant shows decreased binding to the B″-subunit.

The inability of PP1, PP2B, PP5 and PP7 to bind to the PP2A A-subunit may be a consequence of variation at positions corresponding interface amino acid residues in the C-subunit. For example, variation at residues Glu67, Arg70, Arg110 and Asp280 of PP2A C-subunit in related phosphatases may eliminate the ability of these phosphatases to bind to PP2A A-subunit. However, Glu67, Arg70, Arg110 and Asp280 appear to be conserved in PP4 and PP6 at positions corresponding to PP2A C-subunit. In these phosphatases. Lys74 which appears to for a hydrogen bond to Tyr456 of HEAT repeat 12 of the PP2A A-subunit is divergent which may eliminate binding of PP4 or PP6 to PP2A A-subunit.

Both OA and MCLR appear to bind to the same surface pocket on the C-subunit of PP2A despite considerable differences in their chemical identity. This observation is consistent with previous reports that pre-incubation of PP2A with OA prevents binding of MCLR. The binding pocket appears to be located just above the two active site Mn atoms in the of PP2A C-subunit, and an almost identical set of amino acids in the C-subunit of PP2A appears to mediate interactions with both inhibitors. For example, the guanidium group of Arg89 donates two hydrogen bonds to oxygen atoms in OA and MCLR that are in different positions (compare FIG. 4A and FIG. 4B). Similarly, Tyr265 appears to form hydrogen bonds in both toxin-bound complexes. On one end of the binding pocket, four amino acids in the C'-subunit of PP2A, Gln122, Ile123, His191 and Trp200, appear to form a hydrophobic cage, which accommodates a long hydrophobic Adda side chain in MCLR and a hydrophobic portion of OA. On the opposite end of the binding pocket, Leu243, Tyr265, Cys266, Arg268, and Cys269 appear to form multiple van der Waals interactions with a separate hydrophobic portion of OA and MCLR. Interactions between PP2A and MCLR may additionally be strengthened by a covalent linkage between the Sγ atom of Cys269 and the terminal carbon atom of an MCLR side chain (FIG. 4B).

As described above, OA may inhibit PP2A more effectively than PP1 although PP2A and PP1 share approximately 50 percent sequence identity. FIG. 4C shows a comparison overlay of the structure of OA bound to PP2A core with the previously determined structure of OA bound to PP1. Although many residues of PP2A that recognize OA are conserved in PP1, the hydrophobic cage of the PP2A C-subunit that accommodates the hydrophobic portion of OA is absent in PP1 (circled). For example, His191, which resides on the loop between helices α7 and α8 of PP2A C-subunit, appears to contribute to one side of the cage. In contrast, the corresponding residue in PP1, Asp197, along with the corresponding loop are located 4-5 Å further away from the OA molecule. In addition, Gln122 of PP2A, whose aliphatic side chain may contribute to another portion of the hydrophobic cage, is replaced by Ser129 in PP1 which may diminish the capacity of the amino acid at this location on the binding site to mediate van der Waals interactions. The net effect of these substitutions is that PP1 appears to contain an open-ended groove whereas the active site of PP2A appears to contain a hydrophobic cage that may better accommodate the hydrophobic portion of OA as illustrated by FIG. 4D.

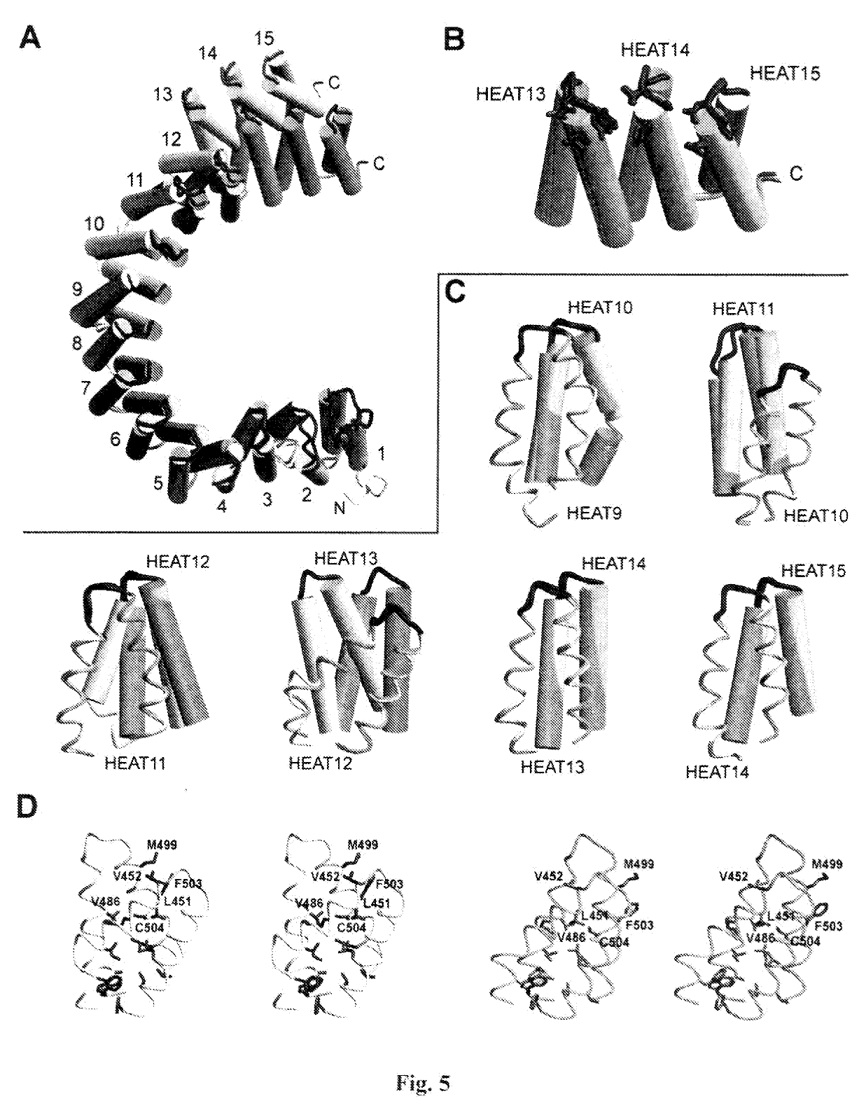

The formation of the PP2A core appears to result in a major conformational switch in the A-subunit. The conformation of the free A-subunit is well defined, with only minor variation in the C-terminal HEAT repeats. HEAT repeats 1-12 of free and PP2A core can be superimposed over one another with an RMSD of 1.25 Å over 444 Cα atoms, and in isolation, HEAT repeats 13-15 of free and PP2A core can be superimposed over one another with an RMSD of 0.49 Å over 114 Cα atoms as illustrated in FIG. 5B. However, as shown in FIG. 5A, when HEAT repeats 1-12 are aligned with each other, HEAT repeats 13-15 can be separated by as much as 20-30 Å. This indicates that a conformational change between HEAT repeats 12 and 13 may be responsible for the observed change in alignment. A pair-wise comparison between the free and the bound A-subunits of all 15 HEAT repeats shows that HEAT repeats 2-10, 14, and 15 appear to exhibit relatively small RMSDs of from about 0.20 Å to about 0.50 Å, and HEAT repeats 11 and 13 appear to exhibit moderate RMSDs of about 0.60 Å. However, HEAT repeat 12 appears to exhibit a much larger conformational change having an RMSD of about 1.4 Å when compared with the other HEAT repeats. As illustrated by FIG. 5C, the relative conformational change from one HEAT repeat to the next reveals that significant conformational changes may occur at the interfaces between repeats 11 and 12 and repeats 12 and 13 which appears to correspond to the interface between the C- and A-subunits.

In fact, the crystallographic data presented herein suggests that packing interactions in the hydrophobic core between adjacent HEAT repeats may be significantly altered due to the binding of the C-subunit. For example, as shown in FIG. 5D, Leu451 appears to interact with Phe503 and Cys504 at the center of the interface between HEAT repeats 12 and 13 in free A-subunit (left panels). However, Leu451 appears to interact with Val486 and Cys504 in the A-subunit of PP2A core (right panels). In addition, Val452, which makes multiple van der Waals contacts at the interface in the free A-subunit (left panels), may rotate completely out of the HEAT repeat interface in the PP2A core (right panels). Similarly alterations may occur within the hydrophobic core between HEAT repeats 11 and 12, albeit to a smaller extent when compared to repeats 12 and 13.

The observed conformational flexibility in the A-subunit of the PP2A core enzyme may have significant functional implications. For example, although the data presented herein only provides information regarding HEAT repeats at the C-subunit/A-subunit interface, without wishing to be bound by theory, conformational changes such as those observed between HEAT repeats 11 and 12, and 12 and 13 may be also be introduced as a result of the binding of regulatory components and/or B-subunits to the A-subunit. Therefore, the conformational flexibility of the extended A-subunit may be an important factor in regulating the catalytic activity of the PP2A holoenzyme.

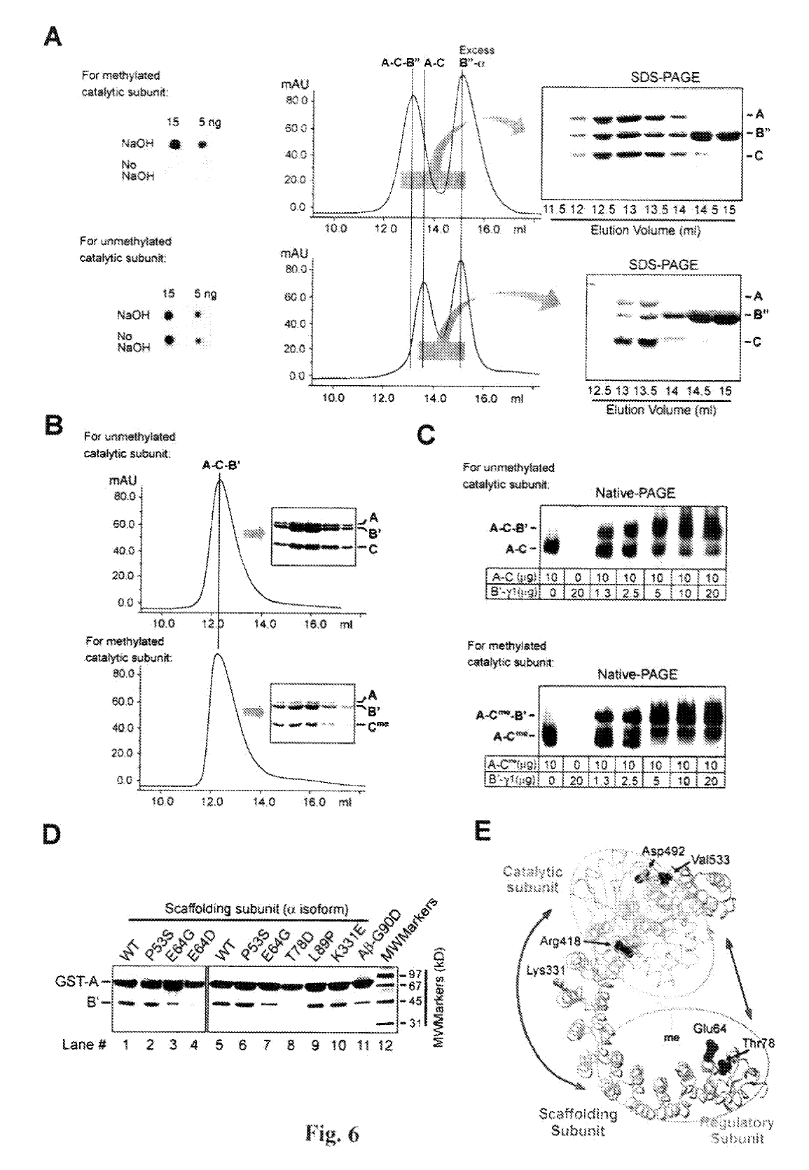

In addition to conformational changes to the A-subunit which may be required for B-subunit binding, B-subunit binding may also require modifications to the C-subunit. For example, methylation of the c-terminal leucine in the C-subunit may be important for the PP2A core interaction with the B- and B′-subunits. To examine whether this requirement applies to B″-subunits, human α isoform of B″-subunit was cloned, expressed, and purified. The PP2A core enzyme was methylated by LCMT in the presence of S-adenosyl methionine (SAM), and the extent of methylation was examined using an antibody that specifically recognizes unmethylated C-terminus of the catalytic subunit of PP2A (FIG. 6A, top left panel). Fully methylated PP2A core was then incubated with excess purified B″-subunit protein, and the resulting mixture was applied to a gel filtration column. The results indicate that methylated PP2A core co-eluted with the B″-subunit in a single peak corresponding to an apparent molecular mass of approximately 160 kDa (FIG. 6A, top right panel), and the elution volume for this peak was smaller than that of PP2A core or B″-subunit alone. These results demonstrate that methylated PP2A core forms a stable holoenzyme with the B″-subunit. In contrast, unmethylated PP2A core enzyme fails to form a stable complex with the B″-subunit as methylated PP2A core eluted from gel filtration column at a volume that is identical to that of the free, unmethylated PP2A core (FIG. 6A, bottom panels).

The conformational flexibility appears to be an intrinsic property of the A-subunit and may be essential to the function of PP2A for at least two reasons. First, conformational flexibility of the A-subunit, which appears to be required for binding to the catalytic subunit, may also be necessary for interacting with other proteins such as B-subunits. For example, PP2A holoenzyme activity may require the B-subunit to be positioned in close proximity to the C-subunit. If the B- and C-subunits bind to opposite ends of the elongated A-subunit, flexibility within the A-subunit may allow the B- and C-subunits to interact as indicated in FIG. 6B. Second, flexibility of the A-subunit may be important for the phosphatase activity of the catalytic subunit. For example, dephosphorylation of target proteins may require a degree of flexibility in the A-subunit. The elongated shape and the relatively loose inter-repeat packing of the HEAT repeats of the A-subunit may be ideally suited to meet such requirements.

Various embodiments of the invention are directed to the atomic coordinates of PP2A core and the use of these atomic coordinates to design or identify molecules that specifically inhibit or activate PP2A core. For example, in some embodiments, the atomic coordinates of PP2A core may used to design and/or screen inhibitor molecules that bind to the PP2A C-subunit in a similar manor as OA and/or MCLR. In other embodiments, the atomic coordinates of PP2A core may be used to design and/or screen inhibitor molecules that bind to the A-subunit and, for example, inhibit the ability of the C-subunit of the PP2A core or the B-subunit of the PP2A holoenzyme to bind to the A-subunit. In further embodiments, the atomic coordinates of PP2A core may be used to design and/or screen molecules that inhibit the flexibility of the A-subunit, such that a C-subunit and a B-subunit may not contact each other or a substrate protein cannot be brought into contact with the active site of the C-subunit. In still other embodiments, the atomic coordinates of PP2A core may be used to design and/or screen activators of PP2A core by, for example, increasing the affinity of the C-subunit for the A-subunit or inducing a bend in the A-subunit that allows C- and B-subunits to interact.

Embodiments encompassing the design and/or screening of molecules that inhibit PP2A activity may include inhibiting the activity PP2A C-subunit and/or inhibiting the ability of the PP2A C-subunit to bind to other components of PP2A core or PP2A holoenzyme. For example, in various embodiments, binding of an inhibitor molecule may mimic OA or MCLR binding thereby selectively reducing or eliminating the catalytic activity of the PP2A C-subunit. In other embodiments, binding of an inhibitor molecule may selectively reduce or eliminate the activity of PP2A core by reducing the ability of the C-subunit to bind the A-subunit by, for example, interrupting the binding interface between the C-subunit and the A-subunit, reducing or eliminating the phosphorylation of the C-subunit, or inhibiting contact or binding of the C-subunit and the B-subunit. In still other embodiments, binding of an inhibitor molecule may reduce or eliminate modifications to the C-subunit, such as, for example, phosphorylation or methylation by inhibiting binding or activity of activating phosphorylases and/or methyl transferases. In additional embodiments, the atomic coordinates of PP2A core described herein may be used to design and/or screen molecules that activate PP2A catalytic activity by, for example, stimulating activating phosphorylation and/or methylation or mimicking the binding of the B-subunit to the C-subunit in the absence of indigenous B-subunit.

Such inhibitors of the PP2A C-subunit may be designed or screened using any method known in the art. For example, in certain embodiments, the atomic coordinates of the OA and MCLR binding site on PP2A C-subunit may be identified, reconstituted and/or isolated in silico and used to design or screen molecules to identify molecules that may fit within the OA and MCLR binding site whereby compounds identified or designed substantially mimic the shape, size, and/or charge of OA or MCLR. In one such embodiment, the portion of the C-subunit used to design and/or screen OA or MCLR mimetics may include at least a portion amino acids 25-288 which make up α-helices 1-9, β-strands 1-14, and intervening loops which make up the OA or MCLR binding site. In another such embodiment, the atomic coordinates of OA, MCLR or a combination of both inhibitors may be used to design and/or screen for other inhibitors.

In other embodiments, a portion of the atomic coordinates of the PP2A C-subunit encompassing the binding interface with the A-subunit or one or more B-subunits may be identified, reconstituted and/or isolated in silico and used to design or screen for molecules that may interrupt interactions between the C-subunit and the A-subunit or one or more B-subunits by binding to the interface. In one such embodiment, the portion of the C-subunit used to design and/or screen inhibitors that bind at the interface region and interrupt binding between the C-subunit and the A-subunit may include at least a portion of amino acids 25-115 and 258-294 which make up α-helices 1-3, β-strands 1-4 and 12-14, and the intervening loops, and the portion of the C-subunit used to design and/or screen inhibitors that bind at the interface region and interrupt binding between the C-subunit and the B-subunit may include at least a portion of amino acids 117-147 and 302-309 which make up α-helices 5 and 6, β-strand 4, and intervening loops. In another such embodiment, portions of the A-subunit and B-subunit that make up the interface between the C-subunit and the A-subunit or one or more B-subunits may be used to design and/or screen molecules that bind to the C-subunit and interrupt the interface. For example, an inhibitor may be designed or screened to mimic at least a portion of amino acids 395-589 which make up HEAT repeats 11-15 of the A-subunit which appear to contact the C-subunit in the PP2A core structure presented above.

In still other embodiments, a portion of the C-subunit may be identified that encompasses a binding interface for an activating protein, such as, for example, a phosphatase or methyl transferase, and this portion of the C-subunit may be reconstituted and/or isolated in silico and used to identify molecules that may interrupt interactions with such activating proteins, thereby inhibiting activation of the C-subunit. For example, the portion of the C-subunit encompassing the area surrounding C-terminal leucine of the C-subunit may be used to design and/or screen molecules that may bind to this portion of the C-subunit and eliminate or reduce the binding of a methyl transferase which may methylate the C-terminal leucine thereby inhibiting the activation of the C-subunit by, for example, inhibiting the association of the C-subunit with a B-subunit.

Other embodiments of the invention include molecules designed and screened to bind to the A-subunit and inhibit various aspects of A-subunit activity thereby inhibiting PP2A core as a whole. For example, in one embodiment, an inhibitor may be designed or molecules may screened and identified that binds to the A-subunit in a similar manor to the C-subunit and or one or more B-subunits. Such a molecule may interrupt or eliminate binding of the C-subunit or one or more B-subunits to the A subunit thereby inhibiting assembly of the PP2A core. In another embodiment, an inhibitor may be designed or a molecule may screened and identified that inhibits or reduces the flexibility of the A-subunit thereby, for example, reducing or eliminating the ability of the A-subunit to bring the C-subunit and one or more B-subunits or other regulatory or substrate proteins into contact.

In certain embodiments, an inhibitor may be designed or molecules may be screened and identified that bind to at least a portion of the A-subunit at the A-subunit/C-subunit interface. For example, in one embodiment, a portion of the A-subunit encompassing at least a section of HEAT repeats 11-15 may be used. Similarly, in some embodiments, an inhibitor may be designed or molecules may be screened and identified that bind to a portion of the A-subunit at the A-subunit/B-subunit interface. For example, in one embodiment, a portion of the A-subunit encompassing HEAT repeats 1-8 may be used, and in another embodiment, the portion of the A-subunit may include at least a portion of amino acids 41-320 which make up HEAT repeats 2-8.

Embodiments including the design or screening of inhibitors which reduce or eliminate flexibility of the A-subunit may include designing or screening any number of compounds which interact with the A-subunit in any number of ways. For example, in some embodiments such an inhibitor may bind between one or more HEAT repeats limiting the movement of these HEAT repeats. In other embodiments, such a compound may generally bind to the A-subunit and various contacts made by the compound may reduce or eliminate the ability of the A-subunit to flex. In still other embodiments, such a compound may interact with one or more consecutive HEAT repeats reducing or eliminating the ability of the portion of the A-subunit encompassing these HEAT repeats to flex thereby reducing the overall flexibility of the A-subunit. In still other embodiments, a compound may bind to one or more HEAT repeats and induce a bend in the A-subunit which may, for example, activate PP2A catalytic activity.

In any of the embodiments described above, a designed or identified inhibitor molecule may have a three-dimensional structure corresponding to at least a portion of PP2A core. For example, an inhibitor may be identified by applying a three-dimensional modeling algorithm to the at least a portion of the atomic coordinates of the PP2A core encompassing, for example, a region of the C-subunit where the inhibitor binds or a region of one or more subunits involved in an interface with another subunit, substrate or regulatory protein and electronically screening stored spatial coordinates of candidate compounds against the atomic coordinates of the PP2A core. Candidate compounds that are identified as substantially complementary to the portion of the PP2A core modeled, or designed to be substantially complementary to the portion of the PP2A core modeled. Candidate compounds so identified may be synthesized using known techniques and then tested for the ability to bind to PP2A core. A compound that is found to effectively bind the PP2A core may be identified as an “inhibitor” of PP2A core if it can then be shown that the binding of the compound reduces or eliminates the activity of the PP2A core. Such “inhibitors” may then be used to modulate the activity of PP2A in vitro or in vivo. In still other embodiments, such “inhibitors” of PP2A may be administered to a subject or used as part of a pharmaceutical composition to be administered to individuals in need thereof.

The terms “complementary” or “substantially complementary” as used herein, refers to a compound having a size, shape, charge or any combination of these characteristics that allow the compound to substantially fill contours created by applying an three-dimensional modeling algorithm to a portion of the PP2A core. A compound that substantially fills without overlapping portions of the various elements that make up the PP2A core, even if various portions of the space remain unfilled, may be considered “substantially complementary”.

The terms “similar” or “substantially similar” may be used to describe a compound having a size, shape, charge or any combination of these characteristics similar to a compound known to bind PP2A core. For example, an identified compound having a similar size, shape, and/or charge to OA or MCLR may be considered “substantially similar” to OA or MCLR.

Any inhibitor identified using the techniques described herein, may bind to PP2A with at least about the same affinity the protein which binds at a selected interface or a known inhibitor to a known binding site, and in certain embodiments, the inhibitor may have an affinity for PP2A that is greater than the affinity of the natural or known substrate for PP2A. Thus, such inhibitors may bind to PP2A and inhibit the activity of PP2A, thereby providing methods and compounds for modulating the activity of PP2A. Without wishing to be bound by theory, inhibition of PP2A may reduce or PP2A mediated serine/threonine phosphorylation, and modulating the activity of PP2A may provide the basis for treatment of various cell cycle modulation or proliferative disorders including, for example, cancer and autoimmune disease.

Determination of the atomic coordinates of any portion of the PP2A core may be carried out by any method known in the art. For example, the atomic coordinates provided in embodiments of the invention, or the atomic coordinates provided by other PP2A crystallographic or NMR structures including, but not limited to, crystallographic or NMR data for PP2A core, PP2A holoenzyme or individual A, B or C components of PP2A, may be provided to a molecular modeling program and the various portions of PP2A core described above may be visualized. In other embodiments, two or more sets of atomic coordinates corresponding to various portions of PP2A core may be compared and composite coordinates representing the average of these coordinates may be used to model the structural features of the portion of PP2A core under study. The atomic coordinates used in such embodiments may be derived from purified PP2A core alone or PP2A bound to any B-subunit of PP2A, PP2A holoenzyme, individual A, B or C subunits, or PP2A bound to other regulatory or substrate proteins, accessory proteins, protein fragments or peptides. In general, atomic coordinates defining a three-dimensional structure of a crystal of a PP2A that diffracts X-rays for the determination of atomic coordinates to a resolution of 5 Angstroms or better may be preferred.

Having defined the structural features of PP2A core, mimetics or small molecules substantially complementary to various portions of the PP2A core, such as those described above, may be designed. Various methods for molecular design are known in the art, and any of these may be used in embodiments of the invention. For example, in some embodiments, compounds may be specifically designed to fill contours of a portion of the PP2A core where an interaction with various subunits of PP2A or other factors interact. In other embodiments, random compounds may be generated and compared to the spatial coordinates such as a portion of PP2A. In still other embodiments, stored spatial coordinates of candidate compounds contained within a database may be compared to the spatial coordinates of a portion of PP2A core. In certain embodiments, molecular design may be carried out in combination with molecular modeling.

In particular embodiments, the atomic coordinates of a subunit bound to another subunit of PP2A or another factor bound to a portion of PP2A, as provided herein, may be used as a basis for mimetic or small molecule inhibitor design or identification. In such embodiments, compounds that mimic the structure of a compound bound to PP2A core and maintain the molecular contacts, such as, for example, hydrogen bonds and van der Waals contacts, may be created or identified such compounds may bind PP2A core and/or inhibit PP2A core activity. In some embodiments, additional features may be added to a compound or portion of a subunit's backbone to create a new compound which provides improved contact between the PP2A and the compound. For example, in one embodiment, a compound may include an additional atom that brings a portion of the compound into closer proximity to a moiety on a portion of PP2A core thereby improving van der Waals interaction or hydrogen bonding potential. In another embodiment, a compound may contain an atom or group of atoms that provide one or more additional hydrogen bond or one or more additional van der Waals contacts.

Methods for performing structural comparisons of atomic coordinates of molecules including those derived from protein crystallography are well known in the art, and any such method may be used in various embodiments to test candidate PP2A core binding compounds for the ability to bind a portion of PP2A core. In such embodiments, atomic coordinates of designed, random or stored candidate compounds may be compared against a portion of the PP2A core structure or the atomic coordinates of OA or MCLR bound to PP2A core. In other such embodiments, a designed, random or stored candidate compound may be brought into contact with a surface of the PP2A core, and simulated hydrogen bonding and/or van der Waals interactions may be used to evaluate or test the ability of the candidate compound to bind the surface of PP2A core. Structural comparisons, such as those described in the preceding embodiments may be carried out using any method, such as, for example, a distance alignment matrix (DAL1), Sequential Structure Alignment Program (SSAP), combinatorial extension (CF) or any such structural comparison algorithm. Compounds that appear to mimic a portion of the PP2A core structure under study or a compound known to bind PP2A core, such as, for example, OA or MCLR, or that are substantially complementary and have a likelihood of forming sufficient interactions to bind to PP2A core may be identified as a potential PP2A core binding compound.

In some embodiments, compounds identified as described above may conform to a set of predetermined variables. For example, in one embodiment, the atomic coordinates of an identified PP2A core binding compound when compared with a native PP2A core binding compound or a subunit of PP2A using one or more of the above structural comparison methods may deviate from a by a RMSD of less than about 10 angstroms. In another embodiment, the identified PP2A core binding compound may include one or more specific structural feature known to exist in a native PP2A core binding compound or a subunit of PP2A core, such as, for example, a surface area, shape, charge distribution over the entire compound or a portion of the identified compound.

Compounds identified by the various methods embodied herein may be synthesized by any method known in the art. For example, identified compounds may be synthesized using various solid state or liquid state synthesis methods.

Compounds identified using various methods of embodiments of the invention may be further tested for binding to PP2A core and/or to determine the compound's ability to inhibit activity of PP2A core or modulate the activity of PP2A core by, for example, testing for pTyr activity or testing the candidate compound for binding to PP2A core. Such testing may be carried out by any method. For example, such methods may include contacting a known substrate with an identified compound and detecting binding to PP2A by a change in fluorescence in a marker or by detecting the presence of the bound compound by isolating the PP2A/candidate compound complex and testing for the presence of the compound. In other embodiments, PP2A activity may be tested by, for example, isolating a substrate peptide that has or has not been phosphorylated by PP2A or isolating a PP2A holoenzyme from a PP2A core that has been contacted with the candidate compound. Such methods are well known in the art and may be carried out in vitro, in a cell-free assay, or in vivo, in a cell-culture assay.

Embodiments of the invention also include pharmaceutical compositions including inhibitors that bind PP2A and inhibit PP2A activity or compounds that are identified using methods of embodiments described herein above and a pharmaceutically acceptable carrier or excipient. Such pharmaceutical compositions may be administered to an individual in an effective amount to alleviate conditions associated with PP2A activity.

The invention described herein encompasses pharmaceutical compositions including a therapeutically effective amount of any of an inhibitor in dosage form and a pharmaceutically acceptable carrier, wherein the compound inhibits the phosphotyrosyl or phosphoserosyl activity of PP2A. In another embodiment, such compositions include a therapeutically effective amount of an inhibitor in dosage form and a pharmaceutically acceptable carrier in combination with a chemotherapeutic and/or radiotherapy, wherein the inhibitor inhibits the phosphotyrosyl or phosphoserosyl activity of PP2A, promoting apoptosis and enhancing the effectiveness of the chemotherapeutic and/or radiotherapy. In various embodiments of the invention, a therapeutic composition for modulating PP2A activity can be a therapeutically effective amount of a PP2A inhibitor.

Embodiments of the invention also include methods for treating a patient having a condition characterized by aberrant cell growth wherein administration of a therapeutically effective amount of a PP2A inhibitor is administered to the patient, and the inhibitor binds to PP2A inducing apoptosis within the area of the patient exhibiting aberrant cell growth. The method may further include the concurrent administration of a chemotherapeutic agent, such as, but not limited to, alkylating agents, antimetabolites, anti-tumor antibiotics, taxanes, hormonal agents, monoclonal antibodies, glucocorticoids, mitotic inhibitors, topoisomerase 1 inhibitors, topoisomerase II inhibitors, immunomodulating agents, cellular growth factors, cytokines, and nonsteroidal anti-inflammatory compounds.

The PP2A inhibitors of the invention may be administered in an effective amount. An “effective amount” is an amount of a preparation that alone, or together with further doses, produces the desired response. This may involve only slowing the progression of the disease temporarily, although it may involve halting the progression of the disease permanently or delaying the onset of or preventing the disease or condition from occurring. This can be monitored by routine methods known and practiced in the art. Generally, doses of active compounds may be from about 0.01 mg/kg per day to about 1000 mg/kg per day, and in some embodiments, the dosage may be from 50-500 mg/kg. In various embodiments, the compounds of the invention may be administered intravenously, intramuscularly, or intradermally, and in one or several administrations per day. The administration of inhibitors can occur simultaneous with, subsequent to, or prior to chemotherapy or radiation.

In general, routine experimentation in clinical trials will determine specific ranges for optimal therapeutic effect for each therapeutic agent and each administrative protocol and administration to specific patients will be adjusted to within effective and safe ranges depending on the patient's condition and responsiveness to initial administrations. However, the ultimate administration protocol will be regulated according to the judgment of the attending clinician considering such factors as age, condition and size of the patient, the potency of the PP2A inhibitor administered, the duration of the treatment and the severity of the disease being treated. For example, a dosage regimen of a PP2A inhibitor to reduce cellular proliferation or induce apoptosis can be oral administration of from about 1 mg to about 2000 mg/day, preferably about 1 to about 1000 mg/day, more preferably about 50 to about 600 mg/day, in two to four divided doses. Intermittent therapy (e.g., one week out of three weeks or three out of four weeks) may also be used.

In the event that a response in a subject is insufficient at the initial doses applied, higher doses (or effectively higher doses by a different, more localized delivery route) may be employed to the extent that the patient's tolerance permits. Multiple doses per day are contemplated to achieve appropriate systemic levels of compounds. Generally, a maximum dose is used, that is, the highest safe dose according to sound medical judgment. However, an individual patient may insist upon a lower dose or tolerable dose for medical reasons, psychological reasons or for virtually any other reason.

Embodiments of the invention also include a method of treating a patient with cancer or an autoimmune disease by promoting apoptosis wherein administration of a therapeutically effective amount of one or more PP2A inhibitors, and the PP2A inhibitor inhibit the phosphotyrosyl or phosphoserosyl activity of PP2A. The method may further include concurrent administration of a chemotherapeutic agent including, but not limited to, alkylating agents, antimetabolites, anti-tumor antibiotics, taxanes, hormonal agents, monoclonal antibodies, glucocorticoids, mitotic inhibitors, topoisomerase I inhibitors, topoisomerase II inhibitors, immunomodulating agents, cellular growth factors, cytokines, and nonsteroidal anti-inflammatory compounds.

A variety of administration routes are available. The particular mode selected will depend upon the severity of the condition being treated and the dosage required for therapeutic efficacy. The methods of the invention may be practiced using any mode of administration that is medically acceptable, meaning any mode that produces effective levels of active compounds without causing clinically unacceptable adverse effects. Such modes of administration include, but are not limited to, oral, rectal, topical, nasal, intradermal, inhalation, intra-peritoneal, or parenteral routes. The term “parenteral” includes subcutaneous, intravenous, intramuscular, or infusion. Intravenous or intramuscular routes may be particularly suitable for purposes of the present invention.

In one aspect of the invention, a PP2A inhibitor as described herein, with or without additional biological or chemotherapeutic agents or radiotherapy, does not adversely affect normal tissues while sensitizing aberrantly dividing cells to the additional chemotherapeutic/radiation protocols. While not wishing to be bound by theory because the PP2A inhibitors specifically target PP2A, marked and adverse side effects may be minimized. In certain embodiments, the composition or method may be designed to allow sensitization of the cell to chemotherapeutic agents or radiation therapy by administering the ATPase inhibitor prior to chemotherapeutic or radiation therapy.

The term “pharmaceutically-acceptable carrier” as used herein, means one or more compatible solid or liquid fillers, diluents or encapsulating substances which are suitable for administration into a human. The term “carrier” or “excipient” denotes an organic or inorganic ingredient, natural or synthetic, with which the active ingredient is combined to facilitate the application. The components of the pharmaceutical compositions are also capable of being co-mingled with the molecules of the present invention and with each other, in a manner such that there is no interaction which would substantially impair the desired pharmaceutical efficacy.

The delivery systems of the invention are designed to include time-released, delayed release or sustained release delivery systems such that the delivering of the PP2A inhibitors occurs prior to, and with sufficient time, to cause sensitization of the site to be treated. A PP2A inhibitor may be used in conjunction with radiation and/or additional anti-cancer chemical agents. Such systems can avoid repeated administrations of the PP2A inhibitor compound, increasing convenience to the subject and the physician, and may be particularly suitable for certain compositions of the present invention.

Many types of release delivery systems are available and known to those of ordinary skill in the art including, but not limited to, polymer base systems, such as, poly(lactide-glycolide), copolyoxalates, polycaprolactones, polyesteramides, polyorthoesters, polyhydroxybutyric acid, and polyanhydrides. Microcapsules of the foregoing polymers containing drugs are described in, for example, U.S. Pat. No. 5,075,109. Delivery systems also include non-polymer systems including, for example: lipids including sterols, such as cholesterol, cholesterol esters and fatty acids or neutral fats, such as mono-, di- and tri-glycerides; hydrogel release systems; sylastic systems; peptide based systems: wax coatings; compressed tablets using conventional binders and excipients; partially fused implants and the like. Specific examples include, but are not limited to: erosional systems in which the active compound is contained in a form within a matrix such as those described in U.S. Pat. Nos. 4,452,775, 4,667,014, 4,748,034, and 5,239,660 and diffusional systems in which an active component permeates at a controlled rate from a polymer, such as described in U.S. Pat. Nos. 3,832,253, and 3,854,480. In addition, pump-based hardware delivery systems can be used, some of which are adapted for implantation.

Use of a long-term sustained release implant may be desirable. Long-term release is used herein, and means that the implant is constructed and arranged to deliver therapeutic levels of the active ingredient for at least about 30 days, and preferably about 60 days. Long-term sustained release implants are well-known to those of ordinary skill in the art and include some of the release systems described above.

The pharmaceutical compositions may conveniently be presented in unit dosage form and may be prepared by any of the methods well known in the art of pharmacy. All methods include the step of bringing the active agent into association with a carrier that constitutes one or more accessory ingredients. In general, the compositions may be prepared by uniformly and intimately bringing the active compound into association with a liquid carrier, a finely divided solid carrier, or both and then, if necessary, shaping the product.

Compositions suitable for parenteral administration conveniently include a sterile aqueous preparation of an ATPase inhibitor which is preferably isotonic with the blood of the recipient. This aqueous preparation may be formulated according to known methods using suitable dispersing or wetting agents and suspending agents. The sterile injectable preparation may also be a sterile injectable solution or suspension in a non-toxic parenterally-acceptable diluent or solvent, for example, as a solution in 1,3-butanediol. Among the acceptable vehicles and solvents that may be employed are water, Ringer's solution, and isotonic sodium chloride solution. In addition, sterile fixed oils are conventionally employed as a solvent or suspending medium. For this purpose, any bland fixed oil may be employed including synthetic mono- or di-glycerides. In addition, fatty acids, such as oleic acid, may be used in the preparation of injectables. Carrier formulation suitable for oral, subcutaneous, intravenous, intramuscular, etc. administrations can be found, for example, in Remington's Pharmaceutical Sciences, Mack Publishing Co., Easton, Pa. which is incorporated herein in its entirety by reference thereto.

### EXPERIMENTAL PROCEDURES

### Protein Preparation and Assembly of PP2A Core Enzyme

All constructs and point mutations were generated using a standard PCR-based cloning strategy. Full-length human PP2A A-subunit a (1-589), all missense mutants, and the regulatory 13″-subunit were cloned into pGEX-2T vector (GE Healthcare) and overexpressed in E. coli strain BL21 (DE3). The soluble fraction of the E. coli cell lysate was purified using glutathione resin (Qiagen) and fractionated by ion-exchange chromatography (Source 15Q, Amersham). Full-length human PP2A C-subunit α (1-309) was cloned into the baculovirus transfer vector pVL1392 (Pharmingen) as an N-terminal 8×His-tagged protein. Recombinant baculovirus was generated using the BaculoGold co-transfection kit (Pharmingen). C-subunit was over-expressed in baculovirus-infected Hi-5 suspension culture and purified to homogeneity on a Ni-NTA column (Qiagen) and fractionated by ion-exchange (Source 15Q, Amersham).

PP2A core was assembled by passing purified C-subunit, which was pre-incubated with an excess amount of MCLR or OA, through a stoichiometric amount of GST-A-subunit immobilized on glutathione resin, Assembled PP2A core was released by on-column thrombin cleavage and further purified by ion-exchange chromatography. Phosphatase assays were performed to ensure that there was no remaining activity for the PP2A core bound to the glutathione resin.

### Crystallization and Data Collection

Crystals of the PP2A core were grown by the hanging-drop vapor-diffusion method by mixing the protein complex (˜10 mg/ml) with an equal volume of reservoir solution containing 0.2 mM lithium sulfate, 1.5 M ammonium sulfate, 0.1 M Tris-Cl pH 8.5. Crystals appeared overnight and grew to full-size within three days. Harvested crystals belong to the space group 1222, with a=92.540 Å, b=194.850 Å, and c=201.350 Å. There is one complex per asymmetric unit. Crystals were equilibrated in a cryoprotectant buffer containing reservoir buffer plus 20 (v/v) % glycerol and were flash frozen in a cold nitrogen stream at −170° C. The native crystallographic data set was collected at NSLS beamline X25 and processed using the software Denzo and Scalepack.

### Structure Determination

The structure of PP2A core was determined by molecular replacement. First, C-subunit was located using the program PHASER and the atomic coordinates of a homologous phosphatase PP1 (accession code 1FJM). A-subunit was subsequently located using the atomic coordinates of free A-subunit α (accession code 1B3U). The solution was examined and modified using O and refined using CNS. The structures were refined to 2.6 and 2.8 Å resolution OA and MCLR bound to PP2A core, respectively. The final refined atomic models contain amino acids 6-294 for C-subunit α and residues 9-589 for A-subunit α.

### Methylation of PP2A Core Enzyme by LCMT

LCMT and PP2A core prepared as described above, was incubated on ice at a 1:2 molar ratio. Methylation was initiated by addition of S-adenosyl methionine (SAM) to a final concentration of 0.75 mM. The methylation reaction was carried out at 22° C. and reached completion after 2-3 hours. The methylated PP2A core was purified away from LCMT by anion exchange chromatography.

### Gel Filtration Chromatography

In each assay, 0.5 mg of the assembled and purified PP2A core was incubated with 0.5 mg of the B-subunit at 4° C. for 10 minutes. This mixture was then applied to Superdex-200 (10/30, GE Healthcare) in buffer containing 25 mM Tris. pH 8.0, 150 mM NaCl and 2 mM DTT. The peak fractions were applied to SDS-PAGE and visualized following coomassie blue staining.

The present invention is not limited to the embodiments described and exemplified above, but is capable of variation and modification within the scope of the appended claims.

What is claimed is:

1. A protein phosphatase 2A (PP2A) binding compound comprising a molecule having a three-dimensional structure corresponding to atomic coordinates derived from at least a portion of an atomic model of protein phosphatase 2A (PP2A) core having okadaic acid or microcystin-LR bound thereto, provided that the compound is not okadaic acid or microcystin.

2. The compound of claim 1, wherein the molecule is an inhibitor of protein phosphatase 2A (PP2A).

3. The compound of claim 1, wherein the molecule has a three-dimensional structure corresponding to atomic coordinates of at least a portion of okadaic acid, microcystin-LR or a combination thereof bound to protein phosphatase 2A (PP2A) core; and
wherein the compound makes interactions with the catalytic (C) subunit of protein phosphatase 2A (PP2A) core that correspond to at least a portion of the interactions observed between the catalytic (C) subunit of protein phosphatase 2A (PP2A) core and okadaic acid or microcystin-LR.

4. The compound of claim 3, wherein the molecule binds protein phosphatase 2A (PP2A) at a binding site for okadaic acid and microcystin-LR on the catalytic (C) subunit of PP2A core.

5. The compound of claim 3, wherein the molecule binds to a portion of the catalytic (C) subunit of protein phosphatase 2A (PP2A) core comprising at least a portion of amino acids 25-288 of the catalytic (C) subunit.

6. The compound of claim 1, wherein the molecule has a shape, a charge, a size or combinations thereof substantially corresponding to a portion of protein phosphatase 2A (PP2A) core.

7. The compound of claim 6, wherein the molecule binds to a catalytic (C) subunit of protein phosphatase 2A (PP2A) core or a scaffolding (A) subunit of protein phosphatase 2A (PP2A) core at an interface between the catalytic (C) subunit and the scaffolding (A) subunit.

8. The compound of claim 6, wherein the molecule corresponds to a portion of the catalytic (C) subunit of protein phosphatase 2A (PP2A) core comprising at least a portion of amino acids 24-115, 258-294 or a combination thereof of the catalytic (C) subunit.

9. The compound of claim 6, wherein the molecule corresponds to a portion of a the scaffolding (A) subunit of protein phosphatase 2A (PP2A) core comprising at least a portion of HEAT repeats 11-15.

10. The compound of claim 1, wherein the molecule has a shape, a charge, a size or combinations thereof substantially complementary to a portion of protein phosphatase 2A (PP2A) core.

11. The compound of claim 10, wherein the molecule is substantially complementary to a portion of a scaffolding (A) subunit of protein phosphatase 2A (PP2A) core.

12. The compound of claim 11, wherein the molecule binds a scaffolding (A) subunit of PP2A core and inhibits flexibility of the scaffolding (A) subunit.

13. The compound of claim 10, wherein the molecule is substantially complementary to a portion of a catalytic (C) subunit of protein phosphatase 2A (PP2A) core corresponding to a region of the catalytic (C) subunit where phosphatase 2A phosphatase activator (PTPA) binds.

14. The compound of claim 13, wherein the molecule inhibits modulation of PP2A by phosphatase 2A phosphatase activator (PTPA).

15. The compound of claim 1, wherein the molecule binds to protein phosphatase 2A (PP2A) core with a greater affinity than a naturally occurring substrate.

16. The compound of claim 1, wherein the molecule inhibits protein phosphatase 2A (PP2A) catalyzed tyrosine phosphorylation, serine phosphorylation or a combination thereof.

17. The compound of claim 1, further comprising a pharmaceutically acceptable excipient or carrier.

18. A method for preparing a protein phosphatase 2A (PP2A) core binding compound comprising:
applying a three-dimensional molecular modeling algorithm to the atomic coordinates of at least a portion of protein phosphatase 2A (PP2A) core, a catalytic (C) subunit of protein phosphatase 2A (PP2A) core, or a scaffolding (A) subunit of protein phosphatase 2A (PP2A) core;
determining spatial coordinates of the at least a portion of protein phosphatase 2A (PP2A) core;
electronically screening stored spatial coordinates of candidate compounds against the spatial coordinates of the at least a portion of protein phosphatase 2A (PP2A) core;
identifying candidate compounds that bind to protein phosphatase 2A (PP2A) core; and
synthesizing the identified candidate compound.

19. The method of claim 18, further comprising identifying a molecule has a shape, a charge, a size or combinations thereof substantially complementary to a portion of protein phosphatase 2A (PP2A) core, the catalytic (C) subunit of protein phosphatase 2A (PP2A) core, or the scaffolding (A) subunit of protein phosphatase 2A (PP2A) core.

20. The method of claim 18, wherein the identified candidate compounds that deviate from the atomic coordinates of the at least a portion of protein phosphatase 2A (PP2A) core, the catalytic (C) subunit of protein phosphatase 2A (PP2A) core, or the scaffolding (A) subunit of protein phosphatase 2A (PP2A) core by a root mean square deviation of less than about 10 angstroms.

21. The method of claim 18, further comprising testing identified candidate compounds for binding protein phosphatase 2A (PP2A) core.

22. The method of claim 18, further comprising testing identified candidate compounds for inhibiting protein phosphatase 2A (PP2A) core activity.

23. The method of claim 18, further comprising identifying candidate compounds having a binding affinity for protein phosphatase 2A (PP2A) core, the catalytic (C) subunit of protein phosphatase 2A (PP2A) core, or the scaffolding (A) subunit of protein phosphatase 2A (PP2A) core greater than a naturally occurring substrate.

24. The method of claim 18, further comprising identifying candidate compounds that inhibit tyrosine phosphorylation, serine phosphorylation or a combination thereof catalyzed by protein phosphatase 2A (PP2A) core.

25. The method of claim 18, wherein the atomic coordinates of at least a portion of the protein phosphatase 2A (PP2A) core or the catalytic (C) subunit of protein phosphatase 2A (PP2A) core further comprise okadaic acid or microcystin-LR bound to the protein phosphatase 2A (PP2A) core or the catalytic (C) subunit; and
wherein electronically screening further comprises electronically screening stored spatial coordinates of candidate compounds against atomic coordinates of okadaic acid or microcystin-LR bound to the protein phosphatase 2A (PP2A) core or the catalytic (C) subunit.

26. A pharmaceutical composition comprising:
an effective amount of a compound prepared by the method comprising:
applying a three-dimensional molecular modeling algorithm to the atomic coordinates of at least a portion of protein phosphatase 2A (PP2A) core, a catalytic (C) subunit of protein phosphatase 2A (PP2A) core, or a scaffolding (A) subunit of protein phosphatase 2A (PP2A) core;
determining spatial coordinates of at least a portion of the protein phosphatase 2A (PP2A) core, the catalytic (C) subunit of protein phosphatase 2A (PP2A) core, or the scaffolding (A) subunit of protein phosphatase 2A (PP2A) core;
electronically screening stored spatial coordinates of candidate compounds against the spatial coordinates of at the least a portion of the protein phosphatase 2A (PP2A) core, the catalytic (C) subunit of protein phosphatase 2A (PP2A) core, or the scaffolding (A) subunit of protein phosphatase 2A (PP2A) core;
identifying compounds that mimic the structure of the at least a portion of the protein phosphatase 2A (PP2A) core, the catalytic (C') subunit of protein phosphatase 2A (PP2A) core, or the scaffolding (A) subunit of protein phosphatase 2A (PP2A) core; and
synthesizing the identified candidate compound; and
a pharmaceutically effective excipient or carrier.

27. The pharmaceutical composition of claim 26, wherein the molecule binds to protein phosphatase 2A (PP2A) core, the catalytic (C) subunit of protein phosphatase 2A (PP2A) core, or the scaffolding (A) subunit of protein phosphatase 2A (PP2A) core.

28. The pharmaceutical composition of claim 26, wherein the atomic coordinates of at least a portion of the protein phosphatase 2A (PP2A) core or the catalytic (C) subunit of protein phosphatase 2A (PP2A) core further comprise okadaic acid or microcystin-LR bound to the protein phosphatase 2A (PP2A) core or the catalytic (C) subunit; and
wherein electronically screening further comprises electronically screening stored spatial coordinates of candidate compounds against atomic coordinates of okadaic acid or microcystin-LR bound to the protein phosphatase 2A (PP2A) core or the catalytic (C) subunit.

29. A method for identifying a carcinogen comprising:
determining the atomic coordinates of a compound;
applying a three-dimensional molecular modeling algorithm to the atomic coordinates of the compound;
applying a three-dimensional molecular modeling algorithm to atomic coordinates of at least a portion of protein phosphatase 2A (PP2A) core, a catalytic (C) subunit of protein phosphatase 2A (PP2A) core, a scaffolding (A) subunit of protein phosphatase 2A (PP2A) core, okadaic acid bound to PP2A core or microcystin-LR bound to PP2A core;
electronically screening atomic coordinates of the compound against the atomic coordinates of at the least a portion of the protein phosphatase 2A (PP2A) core, the catalytic (C) subunit of protein phosphatase 2A (PP2A) core, the scaffolding (A) subunit of protein phosphatase 2A (PP2A) core, okadaic acid bound to PP2A core or microcystin-LR bound to PP2A core; and
identifying the compound as a carcinogen if the compound is substantially similar to the structure of the at least a portion of the protein phosphatase 2A (PP2A) core, the catalytic (C) subunit of protein phosphatase 2A (PP2A) core, the scaffolding (A) subunit of protein phosphatase 2A (PP2A) core, okadaic acid bound to PP2A core or microcystin-LR bound to PP2A core.

30. The method of claim 29, wherein the identified compound deviate from the atomic coordinates of the at least a portion of protein phosphatase 2A (PP2A) core, the catalytic (C) subunit of protein phosphatase 2A (PP2A) core, the scaffolding (A) subunit of protein phosphatase 2A (PP2A) core, okadaic acid bound to PP2A core or microcystin-LR bound to PP2A core by a root mean square deviation of less than about 10 angstroms.

31. The method of claim 29, further comprising testing identified compounds for binding protein phosphatase 2A (PP2A) core.

32. The method of claim 29, further comprising testing identified compounds for inhibiting protein phosphatase 2A (PP2A) core activity.

33. The method of claim 29, further comprising identifying compounds that inhibit tyrosine phosphorylation, serine phosphorylation or a combination thereof catalyzed by protein phosphatase 2A (PP2A) core.

34. The method of claim 29, wherein electronically screening further comprises electronically screening stored spatial coordinates of an identified compound against atomic coordinates of unbound okadaic acid or microcystin-LR.
